(19) United States
(12) Patent Application Publication
(10) Pub. No.: US 2008/0213870 A1
(43) Pub. Date: Sep. 4, 2008

(54) Methods for obtaining modified DNA from a biological specimen

(76) Inventors: Sean Wuxiong Cao, Three Bridges, NJ (US); Jadwiga Katarzyna Markiewicz, Kearny, NJ (US); George A. Green, Newton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/041,206

(22) Filed: Mar. 3, 2008

Related U.S. Application Data
(60) Provisional application No. 60/892,390, filed on Mar. 1, 2007.

(51) Int. Cl. C07H 1/00

(52) U.S. Cl. 435/270; 536/25.41

(57) ABSTRACT

The present invention provides a method for obtaining modified DNA from a biological specimen by obtaining a cell suspension from the specimen, if necessary; passing the cell suspension through a first filter under conditions sufficient to obtain filter-bound cells and suspended DNA; lysing the filter-bound cells under conditions sufficient to release cellular DNA; modifying the DNA bound to the filter under conditions sufficient to release the modified DNA from the filter into a flow-through volume; passing the flow-through volume through a second filter under conditions sufficient to capture the modified DNA to the second filter; and eluting the modified DNA from the second filter.

### STATEMENT REGARDING FEDERALLY SPONSORED RESEARCH OR DEVELOPMENT

No government funds were used to make this invention.

### BACKGROUND OF THE INVENTION

Numerous two-Step DNA extraction/modifications are in use in methylation assays. These methods are expensive and time consuming. For instance, the current method of cell collection requires a centrifuge purchase (roughly $30,000.00), is based on wet chemistry which leads to significant DNA loss during the washing steps, takes at least 4 hours (including 1 h DNA re-hydration) and DNA Extraction and DNA Modification are not performed on the same day (DNA re-hydration is done overnight at 4° C.).

See also, Schoeller et al. (2006); and

http://www.norgenbiotek.com/indexphp?id=urinednakit

One method used by vertebrates and higher plants to regulation gene expression is the methylation of cytosines found in CpG islands located in promoter regions of various genes. In order to study this method of gene regulation, techniques were developed to discriminate methylated cytosines from unmethylated cytosines. One method is to chemically treat DNA in such a way that the cytosines are converted to uracils while 5-methyl-cytosines are not significantly converted. Frommer et al. (1992). A systematic investigation on the critical parameters of the modification procedure has also been made. Grunau et al. (2001). The treated DNA may be used as template for methylation specific PCR (MSP). DNA methylation and methods related thereto are discussed for instance in US patent publication numbers 20020197639, 20030022215, 20030032026, 20030082600, 20030087258, 20030096289, 20030129620, 20030148290, 20030157510, 20030170684, 20030215842, 20030224040, 20030232351, 20040023279, 20040038245, 20040048275, 20040072197, 20040086944, 20040101843, 20040115663, 20040132048, 20040137474, 20040146866, 20040146868, 20040152080, 20040171118, 20040203048, 20040241704, 20040248090, 20040248120, 20040265814, 20050009059, 20050019762, 20050026183, 20050053937, 20050064428, 20050069879, 20050079527, 20050089870, 20050130172, 20050153296, 20050196792, 20050208491, 20050208538, 20050214812, 20050233340, 20050239101, 20050260630, 20050266458, 20050287553 and U.S. Pat. Nos. 5,786,146, 6,214,556, 6,251,594, 6,331,393 and 6,335,165.

DNA modification kits are commercially available, they convert purified genomic DNA with unmethylated cytosines into genomic lacking unmethylated cytosines but with additional uracils. The treatment is a two-step chemical process consisting a deamination reaction facilitated by bisulfite and a desulfonation step facilitated by sodium hydroxide. Typically the deamination reaction is performed as a liquid and is terminated by incubation on ice followed by adding column binding buffer. Following solid phase binding and washing the DNA is eluted and the desulfonation reaction is performed in a liquid. Adding ethanol terminates the reaction and the modified DNA is cleaned up by precipitation. However, both commercially available kits (Zymo and Chemicon) perform the desulfonation reaction while the DNA is bound on the column and washing the column terminates the reaction. The treated DNA is eluted from the column ready for MSP assay. The modification is tedious and has many steps that cause yield loss and increase operator error. All of the available modification procedures begin with purified genomic DNA, which is a tedious process that also has many steps that cause yield loss and increase operator error.

### SUMMARY OF THE INVENTION

The present invention provides a method for obtaining modified DNA from a biological specimen by obtaining a cell suspension from the specimen, if necessary; passing the cell suspension through a first filter under conditions sufficient to obtain filter-bound cells and suspended DNA; lysing the filter-bound cells under conditions sufficient to release cellular DNA; modifying the DNA bound to the filter under conditions sufficient to release the modified DNA from the filter into a flow-through volume; passing the flow-through volume through a second filter under conditions sufficient to capture the modified DNA to the second filter; and eluting the modified DNA from the second filter.

### BRIEF DESCRIPTION OF THE DRAWINGS

FIG. 1 is a flowchart comparison of DNA extraction/modification protocols.

FIG. 2 depicts an individual value plot vs the process used to obtain DNA and assess methylation status of β-actin and GSTPi.

FIG. 3 depicts the results of one-step DNA modification testing.

FIG. 4 depicts the results of one-step DNA modification testing. In FIG. 4, samples were modified using One-Step Protocol (ATL LB) and (+)Ctrl yield comparable B-Actin CT values.

### DETAILED DESCRIPTION OF THE INVENTION

The present invention encompasses a method of obtaining modified DNA from a biological specimen by obtaining a cell suspension from the specimen, if necessary; passing the cell suspension through a first filter under conditions sufficient to obtain filter-bound cells and suspended DNA; lysing the filter-bound cells under conditions sufficient to release cellular DNA; modifying the DNA bound to the filter under conditions sufficient to release the modified DNA from the filter into a flow-through volume; passing the flow-through volume through a second filter under conditions sufficient to capture the modified DNA to the second filter; and eluting the modified DNA from the second filter.

The present invention provides a rapid and efficient method for obtaining bisulfite modified DNA. The method described herein effectively eliminates numerous steps of the previous methods thus reducing possible error while producing superior results. In addition considerable time-savings are also realized.

One-Step DNA Modification Overview
Urine is passed through the syringe filter to collect cells and DNA.

It is possible to utilize syringe or vacuum systems to pass urine through the filter
DNA Extraction is not performed during One-Step DNA Modification Process; crude lysate is modified
Lysis Buffer acts as a denaturing agent for Modification process
One-Step DNA Modification takes less than 4 hours
Virtually no wet chemistry steps

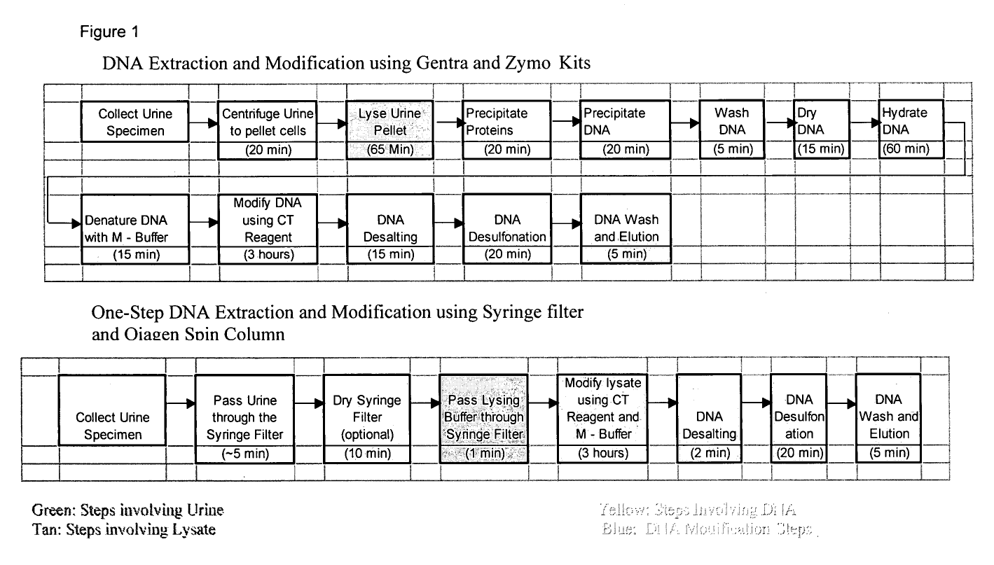

FIG. 1 shows a flowchart comparison of DNA extraction/modification protocols

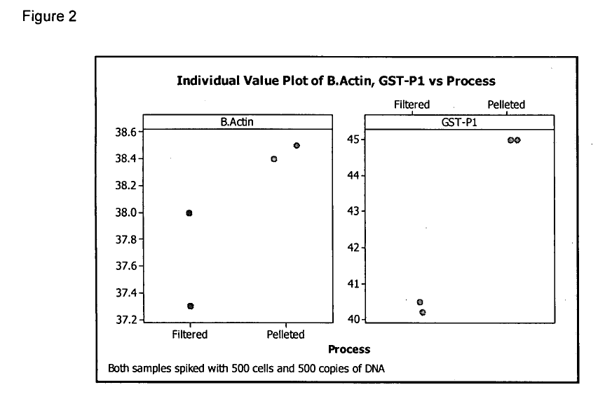

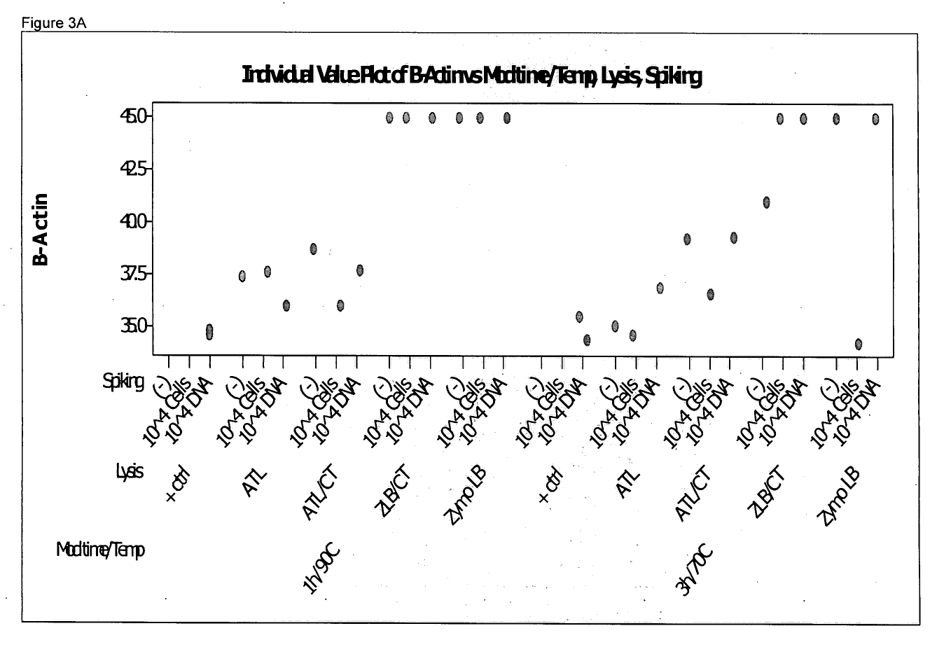

FIG. 2 and Table 1 show initial comparison study data showing that the present method improves not only the time and ease of obtaining modified DNA but provides similar results as previous time-consuming cumbersome methods.

| Urine sample spiking cells/DNA | | | |
|---|---|---|---|
| copies | Processed | β-actin | GSTPi |
| 0/0 | Filtered | 38.7 | 45 |
| 0/0 | Filtered | 37.9 | 45 |
| 500/500 | Filtered | 37.3 | 40.5 |
| 500/500 | Filtered | 38 | 40.2 |
| 0/500 | Filtered | 39.8 | 45 |
| 0/500 | Filtered | 38.7 | 45 |
| 10000/0 | Filtered | 35.2 | 39.3 |
| 10000/0 | Filtered | 35 | 38.8 |
| 500/500 | Pelleted | 38.4 | 45 |
| 500/500 | Pelleted | 38.5 | 45 |

### One-Step DNA Modification Testing Overview

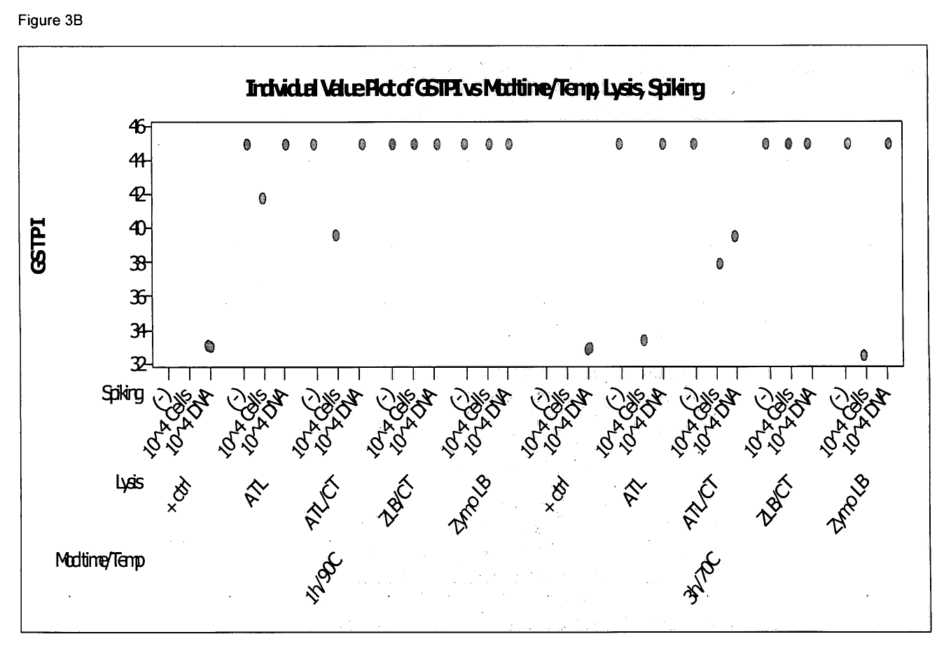

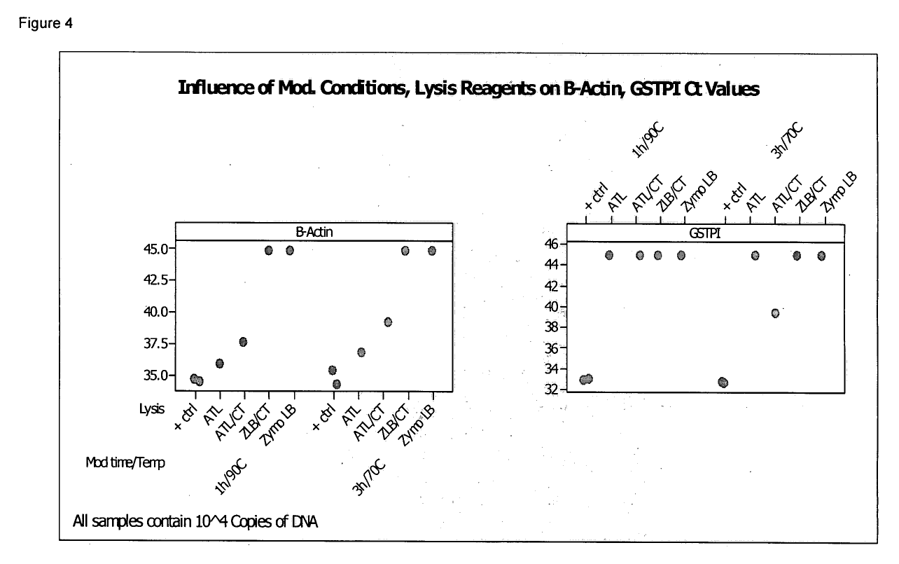

One-step DNA Modification performed using two different lysis buffers (200 μl of LB per syringe filter used)
One-step DNA Modification performed using two different lysis buffers in conjunction with CT Reagent (50 μl of LB and 100 μl of CT per syringe filter used)
Two Modification Reaction Conditions tested (70C/3 h and 90° C./1 h)
10⁴ DNA copies used as a positive control
   The results obtained are shown in Table 2 and FIGS. 3 and 4.

TABLE 2

| | | | | | | | | |
|---|---|---|---|---|---|---|---|---|
| 10⁴ DNA | DNA Dil-N/A | N/A | 3 h/70° C. | 35.5 | 32.9 | 1 h/90° C. | 34.6 | 33 |
| 10⁴ DNA | DNA Dil-N/A | N/A | 3 h/70° C. | 34.4 | 32.8 | 1 h/90° C. | 34.8 | 33.1 |
| (—) | ATL | 200 μl | 3 h/70° C. | 35 | 0 | 1 h/90° C. | 37.4 | 0 |
| 10⁴ cells | ATL | 200 μl | 3 h/70° C. | 34.6 | 33.4 | 1 h/90° C. | 37.6 | 41.8 |
| 10⁴ DNA | ATL | 200 μl | 3 h/70° C. | 36.9 | 0 | 1 h/90° C. | 36 | 0 |
| (—) | Zymo LB | 200 μl | 3 h/70° C. | 0 | 0 | 1 h/90° C. | 0 | 0 |
| 10⁴ cells | Zymo LB | 200 μl | 3 h/70° C. | 34.2 | 32.5 | 1 h/90° C. | 0 | 0 |
| 10⁴ DNA | Zymo LB | 200 μl | 3 h/70° C. | 0 | 0 | 1 h/90° C. | 0 | 0 |
| (—) | ATL/CT | 200 μl | 3 h/70° C. | 39.2 | 0 | 1 h/90° C. | 38.7 | 0 |
| 10⁴ cells | ATL/CT | 200 μl | 3 h/70° C. | 36.6 | 37.9 | 1 h/90° C. | 36 | 39.6 |
| 10⁴ DNA | ATL/CT | 200 μl | 3 h/70° C. | 39.3 | 39.5 | 1 h/90° C. | 37.7 | 0 |
| (—) | ZLC/CT | 200 μl | 3 h/70° C. | 41 | 0 | 1 h/90° C. | 0 | 0 |
| 10⁴ cells | ZLC/CT | 200 μl | 3 h/70° C. | 0 | 0 | 1 h/90° C. | 0 | 0 |
| 10⁴ DNA | ZLC/CT | 200 μl | 3 h/70° C. | 0 | 0 | 1 h/90° C. | 0 | 0 |

The present invention provides a method for obtaining modified DNA from a biological specimen by obtaining a cell suspension from the specimen, if necessary; passing the cell suspension through a first filter under conditions sufficient to obtain filter-bound cells and suspended DNA; lysing the filter-bound cells under conditions sufficient to release cellular DNA; modifying the DNA bound to the filter under conditions sufficient to release the modified DNA from the filter into a flow-through volume; passing the flow-through volume through a second filter under conditions sufficient to capture the modified DNA to the second filter; and, optionally, eluting the modified DNA from the second filter.

DNA can be modified by any method known in the art including, without limitation, methylation, bisulfite modification, biotinylation, restriction endonuclease digestion, fragmentation, fluorescein labeling, sulfurization and phosphorylation.

A biological specimen can be any known in the art including, without limitation, gynecologic smears such as Pap smears; sputum samples; brushings such as bronchial, gastric, or esophageal brushing; washing such as bronchial or gastric washings; fluids such as urine, cerebral spinal fluid, pleural fluid, or abdominal fluid; synovial fluid; fine needle aspiration material; tumor touch samples; and seminal fluid. To form a Pap smear, cells from the cervix or vagina are removed and then examined for cancer to abnormal hormonal conditions. A fine needle aspiration is a minimally invasive method of obtaining cells for biopsy from any area of the body. Sputum samples are mucus or other materials produced by the lining of the respiratory tract, and are sometimes referred to as phlegm, though can include mucus, blood, and pus. Brushing, washing, and fluid samples are collected from various organ sites and used for detection of abnormal cells, malignant cells, and infectious agents.

Preferably, the specimen is in aqueous form for instance, serum, whole blood, plasma, urine, cerebral spinal fluid, tears, semen, aqueous humor and intestinal fluid. Other specimens can be used provided they are reduced to aqueous form such as by agitation, treatment with enzymes such as trypsinase, or homogenization. Such specimens can include any known in the art including, without limitation, bone marrow aspirates, solid organ biopsies, skin samples or biopsies, etc.

The filters used to bind DNA and cells include any known in the art including, without limitation, Zymo ZRC GF Filter, Polyethersulfone (PES), Mixed Cellulose Esters (MCE), Nylon, Fiberglass and DNA-binding filters that are available as a part of DNA Extraction kits.

The following examples are provided to illustrate but not limit the claimed invention. All references cited herein are hereby incorporated herein by reference.

### EXAMPLE 1

### Prior Art Two-Step DNA Extraction/Modification Protocols

I. DNA Extraction from Urine: Gentra Puregene Modified Protocol

### Cell Lysis

1. Centrifuge 50 ml Falcon Tubes containing urine at 3000 g for 15 min @ 4° C.
2. Carefully decant supernatant; leave ˜5 ml of residual supernatant on top of the pellet
3. Centrifuge at 3000 g for 5 min @ 4° C.
4. Discard remaining supernatant using 1 ml pipette. Sample can be stored at −20° C.
5. Add 700 μl of Cell Lysis Solution to the pellet. Pipet up and down to resuspend the pellet. Transfer the sample to 2.0 ml tube.
6. Add 3 μl of Proteinase K solution (20 mg/ml) to the lysate, mix by inverting 25 times and incubate sample for 1 h at 55° C.

### Protein Precipitation

7. Cool sample to room temperature by placing at 20° C. for 10 min
8. Add 300 μl of Protein Precipitation Solution to the lysate
9. Vortex vigorously at high speed for 20 seconds to mix the Protein Precipitation Solution uniformly with the cell lysate
10. Place sample into an ice bath for 5 minutes
11. Centrifuge at (16000 RPM) for 5 minutes. The precipitated proteins will form a tight pellet. Transfer the supernatant to a new 2.0 ml tube and repeat steps 10 to 11

### DNA Precipitation

12. Transfer the supernatant containing the DNA into a clean 2.0 ml microfuge tube
13. To remove any traces of the protein pellet, repeat the centrifugation (16000 RPM for 3 minutes) and transfer the supernatant into a clean 2.0 ml microfuge tube containing 900 μl 100% isopropanol and 2 μl of Glycogen (20 mg/ml)
14. Mix the sample by inverting gently 50 times and keep tube at room temperature for at least 10-15 minutes on the rocker
15. Centrifuge at (16000 RPM) for 5 minutes. The DNA may or may not be visible as a small white pellet, depending on yield
16. Discard supernatant with the 1 ml-pipet
17. Centrifuge at (16000 RPM) for 60 seconds
18. Discard the remaining supernatant with the 100 μl-pipet
19. Add 900 μl 70% ethanol and invert the tube 10 times to wash the DNA pellet
20. Centrifuge at (16000 RPM) for 1 minute
21. Discard ethanol with the 1 ml-pipet
22. Centrifuge at (16000 RPM) for 60 seconds
23. Discard the remaining supernatant with the 100 μl pipet
24. Allow pellet to air dry 10-15 minutes. (Drying Oven)

### DNA Hydration

25. Add 45 μl LoTE buffer
26. Rehydrate DNA by incubating at 65° C. for 1 hour shaking at 1100 rpm and overnight at 20° C. shaking at 1100 rpm
27. Store DNA in a clearly labeled tube at −80° C. (at least one hour freezing before using it)

### II. Sodium Bisulfate Conversion: Zymo Research E-Z DNA Methylation Kit Modified Protocol

### DNA Modification

1. Add 5 μl of M-Dilution Buffer directly to the 45 μl DNA sample
2. Mix sample by flicking or pipetting up and down. Spin the sample briefly. Incubate the sample at 37° C. for 15 minutes in a heat block with shaking at 1100 rpm
3. Prepare CT Conversion Reagent by adding 750 μl Baker Water and 210 μl of M-Dilution Buffer. Vortex for 15 min. CT Reagent is light sensitive, so store it in amber tubes and conduct all incubations in the darkness.
4. Add 100 μl of the prepared CT Conversion Reagent (after briefly spinning) to each sample and vortex lightly
5. Spin the sample briefly. Incubate the sample at 70° C. for 3 hour with the heating block (shaking at 1100 rpm) covered with aluminum foil

### Desalting

6. Spin the sample down briefly. Incubate the sample on ice for 10 min
7. Add 400 μl of M-Binding buffer to the sample and mix by pipetting up and down. Load all the supernatant into a Zymo-Spin Column and place column into a 2 ml collection tube
8. Centrifuge at maximum speed for 30 seconds. Discard the flow-through
9. Add 200 μl of M-Wash Buffer to the column
10. Centrifuge at maximum speed for 30 seconds. Discard the flow-through

### Desulfonation, 2^{nd} Desalting and Elution

11. Add 200 μl of M-Desulfonation Buffer to the column incubate at room temperature for 15 minutes
12. Centrifuge at maximum speed for 30 seconds. Discards the flow-through
13. Add 200 μl of M-Wash Buffer to the column
14. Centrifuge at maximum speed for 15-30 seconds
15. Add another 200 μl of M-Wash Buffer to the column
16. Centrifuge at maximum speed for 1 min. Discard the flow-through
17. Place the column into a clean 1.5 ml tube
18. Add 50 μl of M-elution buffer directly to the column matrix. Let the columns stand for 1 min at RT. Centrifuge at maximum speed for 1 minute to elute the DNA
19. Store the eluted DNA at −80° C.

### EXAMPLE 2

### One Step DNA Modification Protocol of the Present Invention

1. Prepare CT Conversion Reagent by adding 750 μl Baker Water and 210 μl of M-Dilution Buffer. Vortex for 15 min. CT Reagent is light sensitive, so store it in amber tubes and conduct all incubations in the darkness
2. Obtain 60 ml Syringe with Luer Lock. Remove Syringe Plunger
3. Connect Syringe Filter with the Syringe via Luer Lock
4. Pour Urine sample into the Syringe
5. Place Plunger in the Syringe. Push Urine through the filter. Alternatively, attach the Syringe Filter to the Vacuum Unit and apply vacuum to filter urine
6. Optional: Apply vacuum for 10 min to dry the filter
7. Detach Syringe Filter from the 60 ml Syringe; Discard 60 ml Syringe
8. Obtain 1 ml Syringe with Luer Lock. Remove Syringe Plunger and connect Syringe Filter with the 1 ml Syringe via Luer Lock
9. Pipet 200 μl of Lysis Buffer into the Syringe. Alternatively, combine 100 μl of Lysis Buffer with 100 μl of CT Conversion Reagent and pipet it into the Syringe. Omit Step 12
10. Place Plunger in the Syringe. Push Lysis Buffer through the filter into 1.5 ml microfuge tube
11. Add 10 μl of M-Dilution Buffer (Zymo Research) to the lysate
12. Add 200 μl of CT Conversion Reagent to the lysate
13. Incubate lysate at 70° C. for 3 hours in the dark at 1100 RPM. Alternatively, incubate lysate at 90° C. for 1 hour in the dark at 1100 RPM
14. Add 250 μl of 100% Ethanol and mix sample by pipetting up and down
15. Add the sample to a Qiagen DNA purification column (QiaAmp Micro DNA purification kit), spin 1 min at 13,200 rpm and empty waste tube
16. Add 500 μl AW1 wash buffer, spin 1 min at 13,200 rpm and empty waste tube
17. Add 200 μl desulfonation buffer (300 mM NaOH, 90% Ethanol: to prepare, combine 1 ml of 3M NaOH and 9 ml of Ethanol), incubate 20 min room temperature, spin 1 min at 13,200 rpm and empty waste tube
18. Add 500 μl AW2 wash buffer, spin 1 min (13,200 rpm), empty waste tube and spin 3 min (13,200 rpm)
19. Elute with 20-25 μl buffer AE, TE, or Nuclease Free Water. Incubate column for 3 min at room temperature and spin 1 min (13,200 rpm) into new 1.5 mL microfuge tube and store at −20° C. or −80° C.

Although the foregoing invention has been described in some detail by way of illustration and example for purposes of clarity of understanding, the descriptions and examples should not be construed as limiting the scope of the invention.

### REFERENCES

Frommer et al. (1992) A genomic sequencing protocol that yields a positive display of 5-methylcytosine residues in individual DNA strands Proc Natl Acad Sci USA 89:1827-31
Grunau et al. (2001) Bisulfite genomic sequencing: systematic investigation of critical experimental parameters Nucl Acids Res 29:E65-5. http://www.norgenbiotek.com/index.php?id=uriniednakit
Oakeley (1999) DNA methylation analysis: a review of current methodologies Pharmacol Thera 84:389-400
Rathi et al. (2003) Aberrant methylation of the HIC1 promoter is a frequent event in specific pediatric neoplasms Clin Cancer Res 9:3674-33678
Rein et al. (1998) Identifying 5-methylcytosine and related modifications in DNA genomes Nucl Acids Res 26:2255-2264
Schoeller et al. (2006) Preliminary mRNA expression profile of tumor markers in spontaneous urine of prostate cancer patients Clin Chem Lab Med 44:A15, P16

1. A method for obtaining modified DNA from a biological specimen comprising the steps of
1) obtaining a cell suspension from the specimen, if necessary;
2) passing the cell suspension through a first filter under conditions sufficient to obtain filter-bound cells and suspended DNA;
3) lysing the filter-bound cells under conditions sufficient to release cellular DNA;
4) modifying the DNA bound to the filter under conditions sufficient to release the modified DNA from the filter into a flow-through volume;
5) passing the flow-through volume through a second filter under conditions sufficient to capture the modified DNA to the second filter; and
6) eluting the modified DNA from the second filter.

2. The method of claim 1 wherein the biological specimen is in aqueous form.

3. The method of claim 2 wherein the biological specimen is selected from the group consisting of serum, whole blood, plasma, urine, cerebral spinal fluid, tears, semen, aqueous humor and intestinal fluid.

4. The method of claim 2 wherein the biological specimen is selected from the group consisting of gynecologic smears such as Pap smears; sputum samples; brushings such as bronchial, gastric, or esophageal brushing; washing such as bronchial or gastric washings; fluids such as urine, cerebral spinal fluid, pleural fluid, or abdominal fluid; synovial fluid; fine needle aspiration material; tumor touch samples; and seminal fluid.

5. The method of claim 1 wherein the filter is selected from the group consisting of Zymo ZRC GF Filter, Polyethersulfone (PES), Mixed Cellulose Esters (MCE), Nylon, Fiberglass and DNA-binding filters that are available as a part of DNA Extraction kits.

6. The method of claim 1 wherein the DNA modification is selected from the group consisting of methylation, bisulfite modification, biotinylation, restriction endonuclease digestion, fragmentation, fluorescein labeling, sulfurization and phosphorylation.
